(19) United States
(12) Patent Application Publication
(10) Pub. No.: US 2025/0188076 A1
(43) Pub. Date: Jun. 12, 2025

(54) SOLID STATE FORMS OF TOLEBRUTINIB AND OF TOLEBRUTINIB SALTS

(71) Applicant: TEVA PHARMACEUTICAL INTERNATIONAL GMBH, Jona (CH)

(72) Inventor: Lorena Kordic, Rab (HR)

(21) Appl. No.: 18/843,819

(22) PCT Filed: Mar. 9, 2023
(86) PCT No.: PCT/US2023/014868

§ 371 Date: Sep. 4, 2024

Related U.S. Application Data
(60) Provisional application No. 63/413,644, filed on Oct. 6, 2022.
(60) Provisional application No. 63/391,342, filed on Jul. 22, 2022.
(60) Provisional application No. 63/325,705, filed on Mar. 31, 2022.
(60) Provisional application No. 63/318,041, filed on Mar. 9, 2022.

(51) Int. Cl. C07D 471/04

(52) U.S. Cl. CPC C07D 471/04

(57) ABSTRACT

The present disclosure encompasses solid state forms of Tolebrutinib and of Tolebrutinib salts, in embodiments crystalline polymorphs of Tolebrutinib and of Tolebrutinib salts, processes for preparation thereof, and pharmaceutical compositions thereof. In particular, the present invention discloses crystalline polymorphs, salts and co-crystals of Tolebrutinib.

### FIELD OF THE DISCLOSURE

The present disclosure encompasses solid state forms of Tolebrutinib and of Tolebrutinib salts, in embodiments crystalline polymorphs of Tolebrutinib and of Tolebrutinib salts, processes for preparation thereof, and pharmaceutical compositions thereof. In particular, the present invention discloses crystalline polymorphs, salts and co-crystals of Tolebrutinib.

### BACKGROUND OF THE DISCLOSURE

Tolebrutinib, 4-amino-3-(4-phenoxyphenyl)-1-[(3R)-1-prop-2-enoylpiperidin-3-yl]imidazo[4,5-c]pyridin-2-one, has the following chemical structure:

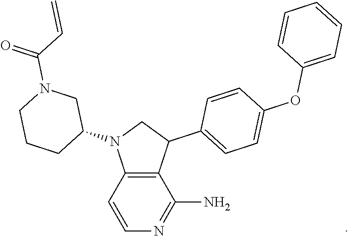 [SMILES: C=CC(=O)N1CCC[C@@H](N2CC(c3ccc(Oc4ccccc4)cc3)c3c2ccnc3N)C1]

Tolebrutinib is a Bruton's tyrosine kinase (BTK) inhibitor, which is believed to reduce the activity of immune cells linked to MS relapses and progression. Tolebrutinib is a small molecule, being investigated for the treatment of relapsing, secondary and primary progressive Multiple Sclerosis (MS), non-relapsing secondary progressive MS, and particularly for reducing or clearing inflammation in MS brain lesions, and for the treatment of myasthenia gravis. The compound is described in U.S. Pat. No. 9,688,676. International Publication Nos. WO 2022/121670 and WO2022223027 disclose crystalline and amorphous forms of Tolebrutinib base. International Publication No. WO2022242740 discloses crystalline forms of Tolebrutinib hydrochloride and Tolebrutinib maleate. International Publication Nos. WO2022257845 and WO2023280132 disclose crystalline forms of Tolebrutinib.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms of Tolebrutinib and of Tolebrutinib salts.

### SUMMARY OF THE DISCLOSURE

The present disclosure provides crystalline polymorphs of Tolebrutinib and of Tolebrutinib salts; in embodiments crystalline polymorphs, salts and/or co-crystals of Tolebrutinib, processes for preparation thereof, and pharmaceutical compositions thereof. These crystalline polymorphs, salts and co-crystals can be used to prepare other forms of Tolebrutinib or Tolebrutinib salts and/or co-crystals thereof.

The present disclosure provides crystalline polymorphs of Tolebrutinib and of Tolebrutinib salts and/or co-crystals of Tolebrutinib for use in the preparation of pharmaceutical compositions and/or formulations for use in medicine, in embodiments for the treatment of Multiple Sclerosis (MS), particularly for relapsing, secondary and primary progressive Multiple Sclerosis (MS), non-relapsing secondary progressive MS, and particularly for reducing or clearing inflammation in MS brain lesions; and for the treatment of myasthenia gravis.

The present disclosure provides crystalline polymorphs of Tolebrutinib and/or of Tolebrutinib salts and/or co-crystals of Tolebrutinib for use in medicine, including for treating MS; in particular, for the treatment of relapsing, secondary and primary progressive MS, non-relapsing secondary progressive MS, and particularly for reducing or clearing inflammation in MS brain lesions; and for the treatment of myasthenia gravis. The present disclosure also encompasses the use of crystalline polymorphs of Tolebrutinib and/or of Tolebrutinib salts and/or co-crystals of Tolebrutinib of the present disclosure for the preparation of pharmaceutical compositions and/or formulations.

In another aspect, the present disclosure provides pharmaceutical compositions comprising any one or a combination of the crystalline polymorphs of Tolebrutinib and/or of Tolebrutinib salts and/or co-crystals of Tolebrutinib according to the present disclosure.

The present disclosure includes processes for preparing the above mentioned pharmaceutical compositions. The processes include combining any one or a combination of the crystalline polymorphs of Tolebrutinib and/or of Tolebrutinib salts and/or co-crystals of Tolebrutinib with at least one pharmaceutically acceptable excipient.

The crystalline polymorphs of Tolebrutinib, Tolebrutinib salts and co-crystals of Tolebrutinib as defined herein and the pharmaceutical compositions or formulations of the crystalline polymorphs of Tolebrutinib and of Tolebrutinib salts and Tolebrutinib cocrystals may be used as medicaments, such as for the treatment of Multiple Sclerosis (MS), particularly for relapsing, secondary and primary progressive Multiple Sclerosis (MS), non-relapsing secondary progressive MS, and particularly for reducing or clearing inflammation in MS brain lesions; and for the treatment of myasthenia gravis.

The present disclosure also provides methods of treating MS by administering a therapeutically effective amount of any one or a combination of the crystalline polymorphs of Tolebrutinib and/or Tolebrutinib salts and/or co-crystals of Tolebrutinib of the present disclosure, or at least one of the above pharmaceutical compositions, to a subject suffering from Multiple Sclerosis (MS), particularly for relapsing, secondary and primary progressive Multiple Sclerosis (MS), non-relapsing secondary progressive MS, and particularly for reducing or clearing inflammation in MS brain lesions; and for the treatment of myasthenia gravis; or otherwise in need of the treatment.

The present disclosure also provides uses of the crystalline polymorphs of Tolebrutinib and/or of Tolebrutinib salts and/or co-crystals of Tolebrutinib of the present disclosure, or at least one of the above pharmaceutical compositions, for the manufacture of medicaments for treating, e.g., Multiple Sclerosis (MS), particularly for relapsing, secondary and primary progressive Multiple Sclerosis (MS), non-relapsing secondary progressive MS, and particularly for reducing or clearing inflammation in MS brain lesions; and for the treatment of myasthenia gravis.

### BRIEF DESCRIPTION OF THE DRAWINGS

FIG. 1 shows a characteristic X-ray powder diffraction pattern (XRPD) of Tolebrutinib Form L1.

FIG. 2 shows a characteristic XRPD of Tolebrutinib Form L2.

FIG. 3 shows a characteristic XRPD of Tolebrutinib Form L3.

FIG. 4 shows a characteristic XRPD of Tolebrutinib hydrochloride salt—Form H1.

FIG. 5 shows a characteristic XRPD of Tolebrutinib mesylate salt—Form M1.

FIG. 6 shows a characteristic XRPD of amorphous Tolebrutinib.

FIG. 7 shows a characteristic XRPD of Tolebrutinib hydrochloride salt—Form H2.

FIG. 8 shows a characteristic XRPD of Tolebrutinib: maleic acid—Form Ma1.

FIG. 9 shows a characteristic XRPD of Tolebrutinib: fumaric acid—Form F1.

FIG. 10 shows a characteristic XRPD of Tolebrutinib:succinic acid—Form S1

FIG. 11 shows a characteristic XRPD of Tolebrutinib:succinic acid—Form S2

FIG. 12 shows a characteristic XRPD of Tolebrutinib hydrobromide salt—Form B1

FIG. 13 shows a characteristic XRPD of Tolebrutinib:succinic acid—Form S1 (0% RH, room temperature).

FIG. 14 shows a characteristic XRPD of Tolebrutinib:succinic acid—Form S1 (100% RH, room temperature).

### DETAILED DESCRIPTION OF THE DISCLOSURE

The present disclosure encompasses solid state forms of Tolebrutinib and of Tolebrutinib salts or Tolebrutinib cocrystals, processes for preparation thereof, and pharmaceutical compositions thereof.

The solid state forms of Tolebrutinib (e.g. Tolebrutinib, Tolebrutinib salts or Tolebrutinib co-crystals) as described in any aspect or embodiment of the present disclosure may be polymorphically pure or substantially free of any other solid state (or polymorphic) forms. As used herein in this context, the expression “polymorphically pure” or “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Tolebrutinib or of Tolebrutinib salts or a co-crystal described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Tolebrutinib or of Tolebrutinib salts or a co-crystal. In some embodiments of the disclosure, the described crystalline polymorph of Tolebrutinib may contain from about 1% to about 20% (w/w), from about 5% to about 20% (w/w), or from about 5% to about 10% (w/w) of one or more other crystalline polymorph of Tolebrutinib.

The solid state forms of Tolebrutinib (e.g. Tolebrutinib, Tolebrutinib salts or Tolebrutinib co-crystals) as described in any aspect or embodiment of the present disclosure may be enantiomerically pure, or substantially free of any other enantiomer, preferably substantially free of the S-enantiomer. As used herein in this context, the expression “substantially free of any other enantiomer” or “substantially free” of a particular enantiomer will be understood to mean that the solid state form contains about 20% (w/w) or less, about 10% (w/w) or less, about 5% (w/w) or less, about 2% (w/w) or less, about 1% (w/w) or less, or about 0% of any other enantiomer or of the particular enantiomer, of the subject compound as measured, for example, by chiral HPLC. Thus, a crystalline polymorph of Tolebrutinib described herein as substantially free of any other enantiomer would be understood to contain greater than about 80% (w/w), greater than about 90% (w/w), greater than about 95% (w/w), greater than about 98% (w/w), greater than about 99% (w/w), or about 100% of the subject enantiomer of crystalline polymorph of Tolebrutinib.

Depending on which other crystalline polymorphs a comparison is made, the crystalline polymorphs of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Tolebrutinib or salt of Tolebrutinib or cocrystal of Tolebrutinib referred to herein as being characterized by graphical data “as depicted in” or “as substantially depicted in” a Figure will thus be to include any crystal forms of Tolebrutinib and of Tolebrutinib salt or Tolebrutinib cocrystal characterized with the graphical data having such small variations, as are well known to the skilled person, in comparison with the Figure.

As used herein, and unless stated otherwise, the term “anhydrous” in relation to crystalline forms of Tolebrutinib, relates to a crystalline form of Tolebrutinib which does not include any crystalline water (or other solvents) in a defined, stoichiometric amount within the crystal. Moreover, an “anhydrous” form would generally not contain more than 1% (w/w), of either water or organic solvents as measured for example by TGA.

“Co-Crystal” or “Co-crystal” as used herein is defined as a crystalline material including two or more molecules in the same crystalline lattice and associated by non-ionic and non-covalent bonds. In some embodiments, the co-crystal includes two or more molecules which are in natural state. In some embodiments, the co-crystal includes two molecules which are in natural state. In embodiments the molar ratio between the active pharmaceutical ingredient (Tolebrutinib) and the coformer (i.e., succinic acid) is between 2:1 and 1:1, and in other embodiments, 2:1.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, unless stated otherwise, the XRPD measurements are taken using copper Kα radiation wavelength 1.5418 Å. XRPD peaks reported herein are measured using CuK α radiation, λ=1.5418 Å, typically at a temperature of 25±3° C.

A thing, e.g., a reaction mixture, may be characterized herein as being at, or allowed to come to “room temperature” or “ambient temperature,” often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

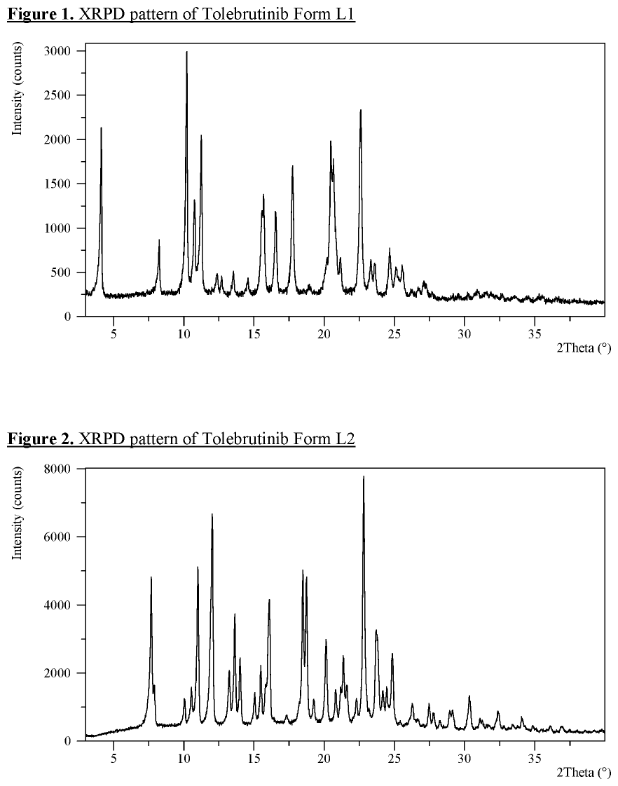

The present disclosure includes a crystalline polymorph of Tolebrutinib, designated L1. The crystalline Form L1 of Tolebrutinib may be characterized by data selected from one or more of the following: an X-ray powder diffraction pattern substantially as depicted in FIG. 1; an X-ray powder diffraction pattern having peaks at 4.1, 10.2, 11.3, 17.8 and 22.6 degrees 2-theta±0.2 degrees 2-theta; and combinations of these data.

Crystalline Form L1 of Tolebrutinib may be further characterized by an X-ray powder diffraction pattern having peaks at 4.1, 10.2, 11.3, 17.8 and 22.6 degrees 2-theta±0.2 degrees 2-theta, and also having any one, two, three, four, or five additional peaks selected from 8.3, 10.8, 16.6, 20.5 and 24.6 degrees 2-theta±0.2 degrees 2-theta.

In one embodiment of the present disclosure, crystalline Form L1 of Tolebrutinib is isolated.

Crystalline Form L1 of Tolebrutinib is anhydrous form.

In another embodiment, the present disclosure includes a crystalline polymorph of Tolebrutinib, designated Form L2. The crystalline Form L2 of Tolebrutinib may be characterized by data selected from one or more of the following: an X-ray powder diffraction pattern substantially as depicted in FIG. 2; an X-ray powder diffraction pattern having peaks at 7.7, 11.0, 12.0, 20.1 and 22.8 degrees 2-theta±0.2 degrees 2-theta; and combinations of these data.

Crystalline Form L2 of Tolebrutinib may be further characterized by an X-ray powder diffraction pattern having peaks at 7.7, 11.0, 12.0, 20.1 and 22.8 degrees 2-theta±0.2 degrees 2-theta, and also having any one, two, three, four, or five additional peaks selected from 13.2, 13.6 14.0, 16.1 and 18.5 degrees 2-theta±0.2 degrees 2-theta.

In one embodiment of the present disclosure, crystalline Form L2 of Tolebrutinib is isolated.

Crystalline Form L2 of Tolebrutinib is anhydrous form.

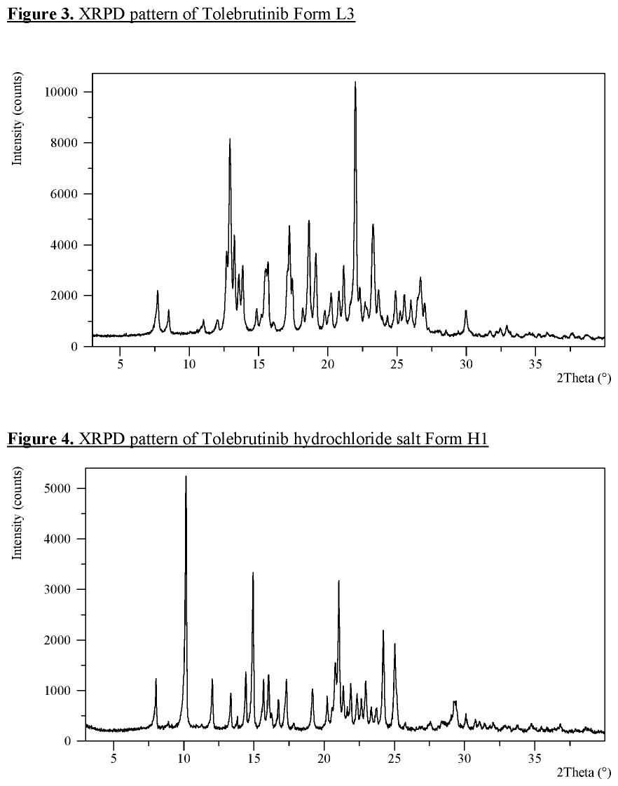

In another embodiment, the present disclosure includes a crystalline polymorph of Tolebrutinib, designated Form L3. The crystalline Form L3 of Tolebrutinib may be characterized by data selected from one or more of the following: an X-ray powder diffraction pattern substantially as depicted in FIG. 3; an X-ray powder diffraction pattern having peaks at 8.5, 12.9, 19.1, 22.0 and 23.3 degrees 2-theta±0.2 degrees 2-theta; and combinations of these data.

Crystalline Form L3 of Tolebrutinib may be further characterized by an X-ray powder diffraction pattern having peaks at 8.5, 12.9, 19.1, 22.0 and 23.3 degrees 2-theta±0.2 degrees 2-theta, and also having any one, two, three or four additional peaks selected from 7.7, 13.9, 18.6, 21.1 and 24.9 degrees 2-theta±0.2 degrees 2-theta.

In one embodiment of the present disclosure, crystalline Form L3 of Tolebrutinib is isolated.

Crystalline Form L3 of Tolebrutinib is anhydrous.

In a further embodiment, the present application discloses a crystalline polymorph of Tolebrutinib hydrochloride salt, designated Form H1. The crystalline Form H1 of Tolebrutinib hydrochloride salt may be characterized by data selected from one or more of the following: an X-ray powder diffraction pattern substantially as depicted in FIG. 4; an X-ray powder diffraction pattern having peaks at 8.0, 10.2, 12.0, 14.9 and 24.2 degrees 2-theta±0.2 degrees 2-theta; and combinations of these data.

Crystalline Form H1 of Tolebrutinib hydrochloride salt may be further characterized by an X-ray powder diffraction pattern having peaks at 8.0, 10.2, 12.0, 14.9 and 24.2 degrees 2-theta±0.2 degrees 2-theta, and also having any one, two, three, four, or five additional peaks selected from 13.4, 14.4, 19.2, 21.1 and 25.0 degrees 2-theta±0.2 degrees 2-theta.

In one embodiment of the present disclosure, crystalline Form H1 of Tolebrutinib hydrochloride salt is isolated.

Crystalline Form H1 of Tolebrutinib hydrochloride salt is anhydrous.

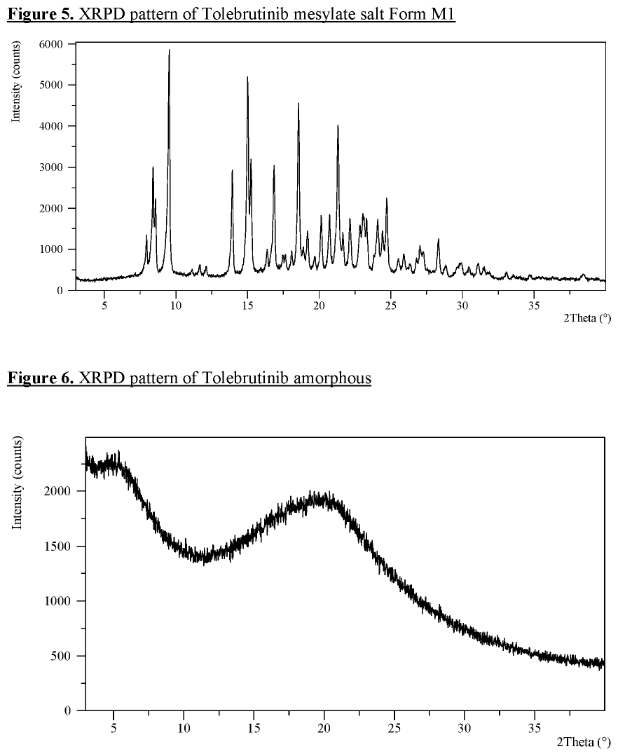

In a further embodiment, the present disclosure includes a crystalline polymorph of Tolebrutinib mesylate salt; designated Form M1. The crystalline Form M1 may be characterized by data selected from one or more of the following: an X-ray powder diffraction pattern substantially as depicted in FIG. 5; an X-ray powder diffraction pattern having peaks at 9.5, 13.9, 16.9, 18.6 and 21.3 degrees 2-theta±0.2 degrees 2-theta; and combinations of these data.

Crystalline Form M1 of Tolebrutinib mesylate salt may be further characterized by an X-ray powder diffraction pattern having peaks at 9.5, 13.9, 16.9, 18.6 and 21.3 degrees 2-theta±0.2 degrees 2-theta, and also having any one, two, three, four, or five additional peaks selected from 8.4, 15.0, 20.1, 20.7 and 22.1 degrees 2-theta±0.2 degrees 2-theta.

In one embodiment of the present disclosure, crystalline Form M1 of Tolebrutinib mesylate salt is isolated.

Crystalline Form M1 of Tolebrutinib mesylate salt is anhydrous.

In a further embodiment, the present application discloses a crystalline polymorph of Tolebrutinib hydrochloride salt, designated Form H2. The crystalline Form H2 of Tolebrutinib hydrochloride salt may be characterized by data selected from one or more of the following: an X-ray powder diffraction pattern substantially as depicted in FIG. 7; an X-ray powder diffraction pattern having peaks at 6.2, 9.6, 11.0, 12.4 and 16.5±0.2 degrees 2-theta±0.2 degrees 2-theta; and combinations of these data.

Crystalline Form H2 of Tolebrutinib hydrochloride salt may be further characterized by an X-ray powder diffraction pattern having peaks at 6.2, 9.6, 11.0, 12.4 and 16.5 degrees 2-theta±0.2 degrees 2-theta, and also having any one, two, three, four, or five additional peaks selected from 15.1, 18.8, 20.2, 22.5 and 25.0 degrees 2-theta±0.2 degrees 2-theta.

In one embodiment of the present disclosure, crystalline Form H2 of Tolebrutinib hydrochloride salt is isolated.

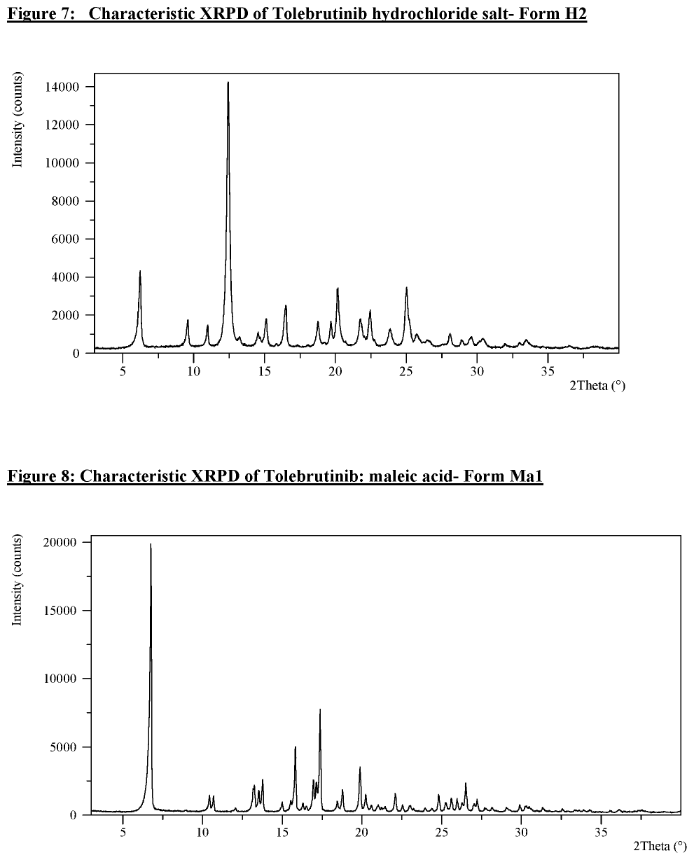

The present disclosure also includes a crystalline polymorph of Tolebritinib: maleic acid; designated Form Ma1. The crystalline Form Ma1 may be characterized by data selected from one or more of the following: an X-ray powder diffraction pattern substantially as depicted in FIG. 8; an X-ray powder diffraction pattern having peaks at 6.8, 15.8, 17.4, 19.9 and 22.1 degrees 2-theta±0.2 degrees 2-theta; and combinations of these data.

Crystalline Form Ma1 of Tolebrutinib: maleic acid may be further characterized by an X-ray powder diffraction pattern having peaks at 6.8, 15.8, 17.4, 19.9 and 22.1 degrees 2-theta±0.2 degrees 2-theta, and also having any one, two, three, four, or five additional peaks selected from 13.8, 18.8, 20.2, 24.8 and 26.5 degrees 2-theta±0.2 degrees 2-theta.

Crystalline Tolebrutinib: maleic acid may be a co-crystal of Tolebrutinib and maleic acid. Alternatively, crystalline Tolebrutinib: maleic acid may be a salt i.e., Tolebrutinib maleate salt. In embodiments the molar ratio between Tolebrutinib and maleic acid is between 2:1 and 1:2, in embodiment 1:1.5 and 1.5:1; in other embodiment about 1:1.5.

Crystalline Form Ma1 of Tolebrutinib: maleic acid may be anhydrous.

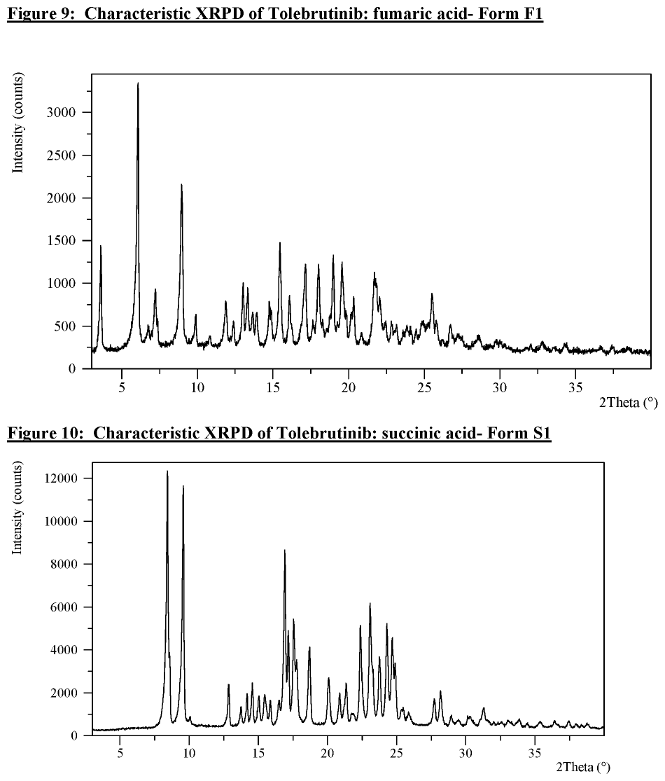

The present disclosure further describes a crystalline polymorph of Tolebrutinib: fumaric acid; designated Form F1. The crystalline Form F1 may be characterized by data selected from one or more of the following: an X-ray powder diffraction pattern substantially as depicted in FIG. 9; an X-ray powder diffraction pattern having peaks at 3.6, 6.1, 9.0, 15.5 and 17.2 degrees 2-theta±0.2 degrees 2-theta; and combinations of these data.

Crystalline Form F1 of Tolebrutinib: fumaric acid may be further characterized by an X-ray powder diffraction pattern having peaks at 3.6, 6.1, 9.0, 15.5 and 17.2 degrees 2-theta±0.2 degrees 2-theta, and also having any one, two, three, four, or five additional peaks selected from 11.9, 13.0, 16.1, 19.0 and 19.6 degrees 2-theta±0.2 degrees 2-theta.

Crystalline Tolebrutinib: fumaric acid may be a co-crystal of Tolebrutinib and fumaric acid. Alternatively, crystalline Tolebrutinib: fumaric acid may be a salt i.e., Tolebrutinib fumarate salt. In embodiments the molar ratio between Tolebrutinib and fumaric acid is between 2:1 and 1:2; in other embodiment about 2:1.

Crystalline Form F1 of Tolebrutinib: fumaric acid may be anhydrous.

The present disclosure further provides crystalline Tolebrutinib:succinic acid. Crystalline Tolebrutinib:succinic acid may be a co-crystal of Tolebrutinib and succinic acid. Alternatively, crystalline Tolebrutinib:succinic acid may be a salt, i.e., Tolebrutinib succinate. Tolebrutinib:succinic acid may be in a molar ratio between about 2:1 to about 1:2 of Tolebrutinib and succinic acid. In one embodiment, Tolebrutinib:succinic acid may be in a 2:1 molar ratio of Tolebrutinib and succinic acid. Preferably, according to any embodiment of the present disclosure, the molar ratio of Tolebrutinib and succinic acid is between about 2:1 to about 1:1 and more preferably 2:1.

Crystalline Tolebrutinib:succinic acid according to any aspect or embodiment of the present disclosure, and preferably in the form of co-crystal of Tolebrutinib with succinic acid, may contain water in an amount of: about 0 to about 7.5 wt %, or about 0 to about 7.0 wt %, about 1.5 wt % to about 6.5 wt %, about 1.8 to 6.2 wt %, or about 2.0 to about 6.0 wt %; and/or the crystalline Tolebrutinib:succinic acid may be a hydrate. Particularly, crystalline Tolebrutinib: succinic acid according to any aspect or embodiment of the disclosure may be a hydrate, preferably containing water in an amount of: about 1.5 wt % to about 6.5 wt %, about 1.8 to 6.2 wt %, or about 2.0 to about 6.0 wt %.

Alternatively, crystalline Tolebrutinib:succinic acid according to any aspect or embodiment of the present disclosure, and preferably in the form of co-crystal of Tolebrutinib with succinic acid, may contain water in an amount of: about 0 to about 2.0 wt %, or about 0.6 to about 1.6 wt %, about 0.8 wt % to about 1.5 wt %, or about 1.0 wt % to about 1.4 wt %.

Alternatively, crystalline Tolebrutinib:succinic acid according to any aspect or embodiment may be a co-crystal of Tolebrutinib with succinic acid, particularly in anhydrous form, and more particularly wherein the crystalline Tolebrutinib:succinic acid contains water in an amount of less than 1 wt %.

In a further embodiment, the present disclosure describes a crystalline polymorph of Tolebrutinib:succinic acid, designated Form S1. The crystalline Form S1 may be characterized by data selected from one or more of the following: an X-ray powder diffraction pattern substantially as depicted in FIG. 10; an X-ray powder diffraction pattern having peaks at 9.6, 14.6, 16.9 and 24.3 degrees 2-theta±0.2 degrees 2-theta; and combinations of these data.

Crystalline Form S1 of Tolebrutinib:succinic acid may be further characterized by an X-ray powder diffraction pattern having peaks at 9.6, 14.6, 16.9 and 24.3 degrees 2-theta±0.2 degrees 2-theta, and also having any one, two, three, four, or five additional peaks selected from 8.6, 12.9, 14.2, 18.7 and 22.4 degrees 2-theta±0.2 degrees 2-theta. Alternatively crystalline Form S1 of Tolebrutinib:succinic acid may be alternatively characterized by an X-ray powder diffraction pattern having peaks at 9.6, 14.6, 16.9 and 24.3 degrees 2-theta±0.2 degrees 2-theta, and also having any one, two, three, or four additional peaks selected from 8.6, 14.2, 18.7 and 22.4 degrees 2-theta±0.2 degrees 2-theta. For example, crystalline Form S1 of Tolebrutinib: succinic acid may be alternatively characterized by an X-ray powder diffraction pattern having peaks at 9.6, 14.6, 16.9 and 24.3 degrees 2-theta±0.2 degrees 2-theta, and also having one additional peak selected from 8.6, 14.2, 18.7 and 22.4 degrees 2-theta±0.2 degrees 2-theta.

In any embodiment, crystalline Form S1 of Tolebrutinib:succinic acid may be further characterized by an X-ray powder diffraction pattern having peaks at 8.6, 9.6, 12.9, 14.2, 14.6, 16.9, 18.7, 22.4, and 24.3 degrees 2-theta±0.2 degrees 2-theta. Alternatively, crystalline Form S1 of Tolebrutinib:succinic acid may be characterized by an X-ray powder diffraction pattern having peaks at 8.6, 9.6, 14.2, 14.6, 16.9, 18.7, 22.4, and 24.3 degrees 2-theta 0.2 degrees 2-theta.

According to any embodiment, the crystalline Form S1 of Tolebrutinib:succinic acid may be an anhydrous form, or may be a hydrate.

Crystalline Form S1 of Tolebrutinib:succinic acid according to any aspect or embodiment of the present disclosure may contain water in an amount of: about 0 to about 7.5 wt %, or about 0 to about 7.0 wt %, about 1.5 wt % to about 6.5 wt %, about 1.8 to 6.2 wt %, or about 2.0 to about 6.0 wt %. Preferably, crystalline form S1 of Tolebrutinib:succinic acid is a hydrate. More preferably, crystalline form S1 Tolebrutinib:succinic acid is hydrate containing water in an amount of: about 1.5 wt % to about 6.5 wt %, about 1.8 to 6.2 wt %, or about 2.0 to about 6.0 wt %.

In any embodiment the molar ratio Tolebrutinib:succinic acid in crystalline Form S1 is 2:1.

Crystalline Form S1 of Tolebrutinib:succinic acid may be a co-crystal of Tolebrutinib and succinic acid (e.g., in a molar ratio of 2:1). Alternatively, crystalline Tolebrutinib:succinic acid may be a salt (i.e., Tolebrutinib succinate). Preferably, crystalline Form S1 of Tolebrutinib:succinic acid as describe in any embodiment disclosed herein is a cocrystal.

Form S1 according to any aspect or embodiment of the present disclosure may be polymorphically pure.

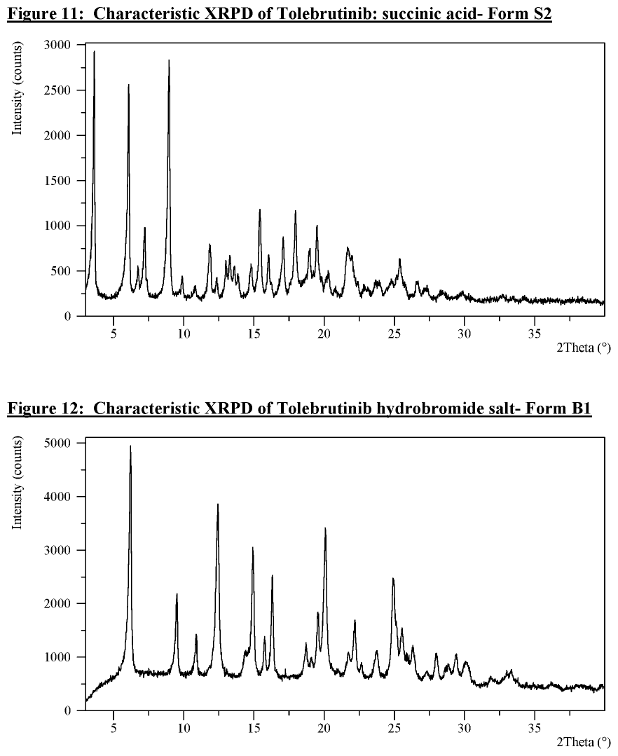

The present disclosure also provides a crystalline polymorph of Tolebrutinib:succinic acid, designated Form S2. The crystalline Form S2 may be characterized by data selected from one or more of the following: an X-ray powder diffraction pattern substantially as depicted in FIG. 11; an X-ray powder diffraction pattern having peaks at 3.6, 6.1 and 9.0 degrees 2-theta 0.2 degrees 2-theta; and combinations of these data.

Crystalline Form S2 of Tolebrutinib:succinic acid may be further characterized by an X-ray powder diffraction pattern having peaks at 3.6, 6.1, 9.0 degrees 2-theta±0.2 degrees 2-theta, and also having any one, two, three, four, five or six additional peaks selected from 11.9, 15.5, 16.0, 17.1, 18.0 and 19.5 degrees 2-theta: 0.2 degrees 2-theta.

The crystalline polymorph S2 of Tolebrutinib:succinic acid may be alternatively characterized by data selected from one or more of the following: an X-ray powder diffraction pattern substantially as depicted in FIG. 11; an X-ray powder diffraction pattern having peaks at 3.6, 6.1, 9.0, 18.0 and 19.5 degrees 2-theta±0.2 degrees 2-theta; and combinations of these data.

Crystalline Form S2 of Tolebrutinib:succinic acid may be further characterized by an X-ray powder diffraction pattern having peaks at 3.6, 6.1, 9.0, 18.0 and 19.5 degrees 2-theta±0.2 degrees 2-theta, and also having any one, two, three, or four additional peaks selected from 11.9, 15.5, 16.0 and 17.1 degrees 2-theta±0.2 degrees 2-theta.

In a further embodiment, crystalline Form S2 of Tolebrutinib:succinic acid may be further characterized by an X-ray powder diffraction pattern having peaks at 3.6, 6.1, 9.0, 11.9, 15.5, 16.0, 17.1, 18.0 and 19.5 degrees 2-theta±0.2 degrees 2-theta.

Crystalline Form S2 of Tolebrutinib:succinic acid may be anhydrous.

Alternatively, crystalline Form S2 of Tolebrutinib:succinic acid according to any aspect or embodiment of the disclosure may be a hydrate form.

Crystalline Form S2 of Tolebrutinib:succinic acid according to any aspect or embodiment may contain water in an amount of: about 0 to about 2.0 Wt %, or about 0.6 to about 1.6 wt %, about 0.8 wt % to about 1.5 wt %, or about 1.0 wt % to about 1.4 wt %.

Preferably, according to any aspect or embodiment, crystalline Form S2 of Tolebrutinib:succinic acid is anhydrous, and more preferably contains less than 1 wt % water.

In any embodiment the molar ratio Tolebrutinib:succinic acid in crystalline Form S2 is 2:1.

Crystalline Form S2 of Tolebrutinib:succinic acid may be a co-crystal of Tolebrutinib and succinic acid (e.g., in a molar ratio of 2:1). Alternatively, crystalline Tolebrutinib:succinic acid may be a salt (i.e., Tolebrutinib succinate). Preferably, crystalline Form S2 of Tolebrutinib:succinic acid as described in any embodiment disclosed herein is a cocrystal.

Form S2 according to any aspect or embodiment of the present disclosure may be polymorphically pure.

The above crystalline polymorphs and co-crystals of Tolebrutinib and of Tolebrutinib salts or Tolebrutinib cocrystals can be used to prepare other crystalline polymorphs of Tolebrutinib, other Tolebrutinib salts or other Tolebrutinib cocrystals, and solid state forms thereof.

In a further embodiment, the present application discloses a crystalline polymorph of Tolebrutinib hydrobromide salt, designated Form B1. The crystalline Form B1 of Tolebrutinib hydrobromide salt may be characterized by data selected from one or more of the following: an X-ray powder diffraction pattern substantially as depicted in FIG. 12; an X-ray powder diffraction pattern having peaks at 6.2, 9.5, 12.4, 15.0 and 16.3 degrees 2-theta±0.2 degrees 2-theta; and combinations of these data.

Crystalline Form B1 of Tolebrutinib hydrobromide salt may be further characterized by an X-ray powder diffraction pattern having peaks at 6.2, 9.5, 12.4, 15.0 and 16.3 degrees 2-theta±0.2 degrees 2-theta, and also having any one, two, three, four, or five additional peaks selected from 10.9, 15.8, 20.1, 22.2 and 24.9 degrees 2-theta±0.2 degrees 2-theta.

In one embodiment of the present disclosure, crystalline Form B1 of Tolebrutinib hydrobromide salt is isolated.

In embodiments, crystalline Form B1 of Tolebrutinib hydrobromide salt is anhydrous.

The present disclosure provides crystalline polymorphs and co-crystals of Tolebrutinib and of Tolebrutinib salts or Tolebrutinib cocrystals for use in the preparation of pharmaceutical compositions including Tolebrutinib or Tolebrutinib salts, or Tolebrutinib cocrystals and/or crystalline polymorphs thereof.

The present disclosure also encompasses the use of crystalline polymorphs of Tolebrutinib and/or Tolebrutinib salts, or Tolebrutinib cocrystals of the present disclosure for the preparation of pharmaceutical compositions of crystalline polymorphs Tolebrutinib and/or Tolebrutinib salts, or Tolebrutinib cocrystals.

The present disclosure includes processes for preparing the above-mentioned pharmaceutical compositions. The processes include combining any one or a combination of the crystalline polymorphs of Tolebrutinib and/or Tolebrutinib salts, or Tolebrutinib cocrystals of the present disclosure with at least one pharmaceutically acceptable excipient.

Pharmaceutical combinations or formulations of the present disclosure contain any one or a combination of the solid state forms of Tolebrutinib or of Tolebrutinib salts, or Tolebrutinib cocrystals of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Tolebrutinib and/or Tolebrutinib salt and/or Tolebrutinib cocrystals and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Tolebrutinib or of Tolebrutinib salt, or Tolebrutinib cocrystal can be administered. Tolebrutinib or Tolebrutinib salt or Tolebrutinib cocrystals as described in any aspect or embodiment herein may be formulated as a solid oral dosage form, such as a capsule or a tablet, or more preferably a tablet. Tolebrutinib or Tolebrutinib salt or Tolebrutinib cocrystals as described in any aspect or embodiment herein may be formulated for administration to a mammal, in embodiments to a human, by injection. Tolebrutinib or Tolebrutinib salt, or Tolebrutinib cocrystal can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Tolebrutinib and of Tolebrutinib salts or Tolebrutinib cocrystals, and the pharmaceutical compositions and/or formulations of Tolebrutinib and of Tolebrutinib salts, or Tolebrutinib cocrystals of the present disclosure, can be used as medicaments.

The present disclosure also provides methods of treating Multiple Sclerosis, particularly for relapsing, secondary and primary progressive Multiple Sclerosis (MS), non-relapsing secondary progressive MS, and particularly for reducing or clearing inflammation in MS brain lesions; and for the treatment of myasthenia gravis, by administering a therapeutically effective amount of any one or a combination of the crystalline polymorphs of Tolebrutinib and/or Tolebrutinib salts, or Tolebrutinib cocrystals of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

Sample after being powdered in a mortar and pestle is applied directly on a silicon plate holder. The X-ray powder diffraction pattern was measured with Philips X'Pert PRO X-ray powder diffractometer, equipped with Cu irradiation source=1.5418 Å, X'Celerator (2.022° 2θ) detector.

### Measurement Parameters:

Scan range: 3-40 degrees 2-theta;
Scan mode: continuous;
Step size: 0.0167 degrees;
Time per step: 37 s;
Sample spin: 30 rpm;
Peak positions were determined without using silicone powder as an internal standard.

### XRPD Method for 0 and 100% RH:

Sample was prepared by lightly grinding the bulk sample with agate mortar and pestle. Prepared sample was then loaded in 0.5 mm transmission cell for Anton Paar TTK 600 non-ambient camera. For 0% relative humidity (“RH”) measurement the sample compartment was purged with dry nitrogen for 4 hours, and for 100% RH measurement the sample compartment was purged with nitrogen gas passing through a bubbler filled with distilled water for 4 hours. Diffracted intensities were collected in transmission mode (Debye-Scherrer geometry) on Panalytical Empyrean diffractometer equipped with Cu K-alpha tube, W/Si graded elliptic focusing mirror for incident beam optics and iCore automatic optic module for diffracted beam optics coupled with PIXcel3D-Medipix3 detector in line scan mode.

Scan parameters: scan range (° 2theta) 5-40, step size (° 2theta) 0.0066, counting time (s) 19.851. All measurements, sample preparation and conditioning were performed at 25° C.

### EXAMPLES

### Preparation of Starting Materials

Tolebrutinib can be prepared according to methods known from the literature, for example U.S. Pat. No. 9,688,676.

### Example 1: Preparation of Amorphous Tolebrutinib

500 mg of Tolebrutinib was dissolved in dichloromethane (5 mL) at room temperature. Obtained solution was fast evaporated on rotavapor. Obtained solid was analyzed by XRPD. Tolebrutinib amorphous was obtained, as shown in FIG. 6.

### Example 2: Preparation of Tolebrutinib Form L1

40 mg of amorphous Tolebrutinib base was exposed to acetone vapors at room temperature for 4 days. Obtained powder was analyzed by XRPD. Tolebrutinib Form L1 was obtained, as shown in FIG. 1.

### Example 3: Preparation of Tolebrutinib Form L1

50 mg of amorphous Tolebrutinib base was suspended in 1 mL of diethoxymethane. Suspension was stirred overnight at room temperature. Obtained powder was filtered off over a blue ribbon filter paper (under vacuum) and analyzed by XRPD. Tolebrutinib Form L1 was obtained.

### Example 4: Preparation of Tolebrutinib Form L2

300 mg of amorphous Tolebrutinib base was suspended in 3 mL of 2-methyltetrahydrofuran/ethyl acetate (9:1) solvent mixture. Suspension was stirred for 3 days at room temperature. Obtained powder was filtered off over blue ribbon filter paper (under vacuum) and analyzed by XRPD. Tolebrutinib Form L2 was obtained, as shown in FIG. 2.

### Example 5: Preparation of Tolebrutinib Form L2

50 mg of amorphous Tolebrutinib base was suspended in 0.5 mL of 2-propanol/acetone (9:1) solvent mixture. Suspension was stirred for four days at room temperature. Obtained powder was filtered off over blue ribbon filter paper (under vacuum) and analyzed by XRPD. Tolebrutinib Form L2 was obtained.

### Example 6: Preparation of Tolebrutinib Form L3

500 mg of amorphous Tolebrutinib base was suspended in 5 mL of diethyl ether. Suspension was stirred four days at room temperature. Obtained powder was filtered off over blue ribbon filter paper (under vacuum) and analyzed by XRPD. Tolebrutinib Form L3 was obtained, as shown in FIG. 3.

### Example 7: Preparation of Tolebrutinib Form L3

300 mg of amorphous Tolebrutinib base was suspended in 3 mL of tert-butyl methyl ether. Suspension was stirred for four days at room temperature. Obtained powder was filtered off over blue ribbon filter paper (under vacuum) and analyzed by XRPD. Tolebrutinib Form L3 was obtained.

### Example 8: Preparation of Tolebrutinib Hydrochloride Salt Form H1

600 mg of amorphous Tolebrutinib base was suspended in 4 mL of acetone. 0.219 mL (2 eq.) of concentrated hydrochloride acid was added to the suspension at room temperature. Obtained suspension was heated to 50° C. for 1 hour and then stirred for two days at room temperature. Obtained powder was filtered off over blue ribbon filter paper (under vacuum) and analyzed by XRPD. Tolebrutinib hydrochloride salt Form H1 was obtained, as shown in FIG. 4.

### Example 9: Preparation of Tolebrutinib Mesylate Salt Form M1

200 mg of amorphous Tolebrutinib base was suspended in 1.5 mL of 2-butanol. 0.06 mL (2 eq.) of methanesulfonic acid was added to the suspension at room temperature. Obtained suspension was stirred for four days at room temperature. Obtained powder was filtered off over blue ribbon filter paper (under vacuum) and analyzed by XRPD. Tolebrutinib mesylate salt Form M1 was obtained, as shown in FIG. 5.

### Example 10: Preparation of Tolebrutinib Hydrochloride Salt Form H2

500 mg of amorphous Tolebrutinib base was dissolved in 6 mL of tetrahydrofuran at 70° C. Heating is turned off and solution was left to cool to room temperature. HCl solution in 2-propanol (5-6M, 440 μL) was added to the solution at room temperature and crystallization occurred. Obtained powder was filtered off over blue ribbon filter paper (under vacuum) and analyzed by XRPD. Tolebrutinib hydrochloride Form H2 was obtained, as shown in FIG. 7.

### Example 11: Preparation of Tolebrutinib:Maleic Acid—Form Ma1

250 mg of amorphous Tolebrutinib base and 120 mg of maleic acid were suspended in 2 mL of methyl isobutyl ketone. Suspension was left for stirring overnight at room temperature. Obtained powder was filtered off over blue ribbon filter paper (under vacuum) and analyzed by XRPD. Tolebrutinib: maleic acid—Form Ma1 was obtained, as shown in FIG. 8.

### Example 12: Preparation of Tolebrutinib:Fumaric Acid—Form F1

300 mg of amorphous Tolebrutinib base and 39 mg of fumaric acid were dissolved in 3 mL of 2-buthanol at room temperature. After a few minutes crystallization occurred. Suspension was left for stirring overnight. Obtained powder was analyzed by XRPD. Tolebrutinib: fumaric acid—Form F1 was obtained, as shown in FIG. 9.

### Example 13: Preparation of Tolebrutinib:Succinic Acid—Form S1

600 mg of amorphous Tolebrutinib base and 270 mg of succinic acid were dissolved in 7 mL of acetonitrile at room temperature. After a few minutes crystallization occurred. Suspension was left for stirring for 20 days. Sample was filtered over blue ribbon filter paper (under vacuum) and dried in a vacuum oven for 2 hours at 50° C. Obtained powder was analyzed by XRPD. Crystalline Form S1 of Tolebrutinib:succinic acid was obtained, as shown in FIG. 10.

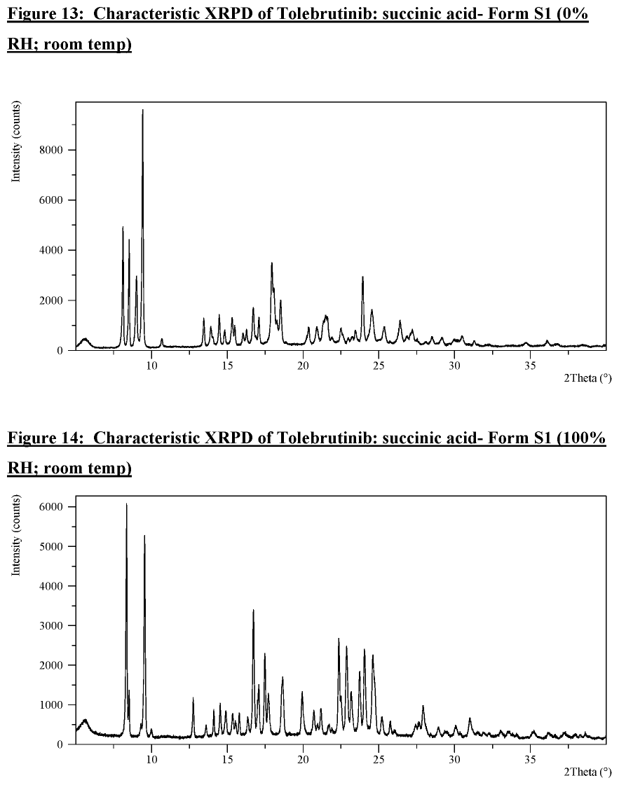

The sample was also analyzed by a non-ambient XRPD: at room temperature at 0% RH (FIG. 13); and at room temperature at 100% RH (FIG. 14).

### Example 14: Preparation of Tolebrutinib:Succinic Acid—Form S2

220 mg of amorphous Tolebrutinib base and 30 mg of succinic acid were dissolved in 5 mL of acetonitrile at room temperature. After a few minutes crystallization occurred. Suspension was left for stirring for 2 hours. Sample was filtered under vacuum and analyzed by XRPD. Form S2 of Tolebrutinib:succinic acid was obtained, as shown in FIG. 11.

### Example 15: Preparation of Tolebrutinib Hydrobromide Salt—Form B1

600 mg of amorphous Tolebrutinib base was dissolved in 6 mL of tetrahydrofuran at 70° C. Obtained solution was left to cool to room temperature. Then 0.528 mL aqueous solution of hydrobromide (3.7 eq) was added. After a few minutes crystallization occurred. Suspension was left for stirring overnight at room temperature. Sample was filtered over blue ribbon filter paper (under vacuum) and dried in a vacuum oven for 2 hours at 70° C. Obtained powder was analyzed by XRPD. Form B1 of Tolebrutinib hydrobromide salt was obtained, as shown in FIG. 12.

### Example 16

### Storage Stability at Different Relative Humidities

Samples of Tolebrutinib:succinic acid were subjected to conditions of different relative humidities at ambient temperature or at 40° C. XRPD analysis was performed on the samples after 7 days or after 1 month. The results are presented in Tables 1-4 below:

TABLE 1

| Tolebrutinib: | | | | | |
|---|---|---|---|---|---|
| succinic acid | Relative humidity (1 month, room temperature) | | | | |
| Form S1 | 20% | 40% | 60% | 80% | 100% |
| XRPD analysis | No | No | No | No | No |
| results | polymorphic | polymorphic | polymorphic | polymorphic | polymorphic |
| | conversion | conversion | conversion | conversion | conversion |

TABLE 2

| Tolebrutinib: | | |
|---|---|---|
| succinic acid | Relative humidity (1 month, 40° C.) | |
| Form S1 | 75% | 100% |
| XRPD analysis results | No polymorphic conversion | No polymorphic |
| | | conversion |

TABLE 3

| | |
|---|---|
| Tolebrutinib: | |
| succinic acid | Relative humidity (7 days, room temperature) |

| Form S2 | 80% | 100% |
|---|---|---|
| XRPD analysis | No polymorphic | No polymorphic |
| results | conversion | conversion |

TABLE 4

| Tolebrutinib: | Relative humidity |
|---|---|
| succinic acid | (7 days, 40° C.) |
| Form S2 | 75% |
| XRPD analysis results | No polymorphic conversion |

These results demonstrate that Tolebrutinib:succinic acid is especially stable to high and low relative humidity conditions and is particularly suitable for use in pharmaceutical dosage forms.

### Example 17: Solubility Comparison of Forms S1 and S2 with Form CSI as Disclosed in WO 2022/121670

The solubilities of Tolebrutinib:succinic acid and Tolebrutinib Form CSI, were compared in water and aqueous acetate buffer at pH 4.5. Tolebrutinib:succinic acid Forms S1 and S2 were both found to have significantly greater solubilities (>0.4 mg/mL) compared with Tolebrutinib Form CSI (<0.2 mg/mL).

The results demonstrate the significantly superior solubility of Tolebrutinib:succinic acid compared with Tolebrutinib Form CSI. Therefore, Tolebrutinib:succinic acid is expected to show greater bioavailability compared with Tolebrutinib Form CSI as disclosed in WO 2022/121670.

1. A crystalline Tolebrutinib:succinic acid.

2. The crystalline Tolebrutinib:succinic acid according to claim 1 which is a co-crystal of Tolebrutinib and succinic acid or a salt of Tolebrutinib with succinic acid, or a co-crystal of Tolebrutinib and succinic acid.

3. The crystalline Tolebrutinib:succinic acid according to claim 1, wherein the molar ratio of Tolebrutinib and succinic acid is between about 2:1 to about 1:1.

4. The crystalline form of Tolebrutinib:succinic acid according to claim 1, designated Form S1, which is characterized by data selected from:
(a) an X-ray powder diffraction pattern substantially as depicted in FIG. 10; or
(b) an X-ray powder diffraction pattern having peaks at 9.6, 14.6, 16.9 and 24.3 degrees 2-theta±0.2 degrees 2-theta.

5. The crystalline Tolebrutinib:succinic acid according to claim 1, which is characterized by an X-ray powder diffraction pattern having peaks at 9.6, 14.6, 16.9 and 24.3 degrees 2-theta±0.2 degrees 2-theta, and also having any one, two, three, four, or five additional peaks selected from 8.6, 12.9, 14.2, 18.7 and 22.4 degrees 2-theta±0.2 degrees 2-theta.

6. The crystalline Tolebrutinib:succinic acid according to claim 1, which is characterized by an X-ray powder diffraction pattern having peaks at 9.6, 14.6, 16.9 and 24.3 degrees 2-theta±0.2 degrees 2-theta, and having any one, two, three, or four additional peaks selected from 8.6, 14.2, 18.7 and 22.4 degrees 2-theta±0.2 degrees 2-theta; or which is characterized by an X-ray powder diffraction pattern having peaks at 9.6, 14.6, 16.9 and 24.3 degrees 2-theta±0.2 degrees 2-theta, and having any one additional peak selected from 8.6, 14.2, 18.7 and 22.4 degrees 2-theta±0.2 degrees 2-theta.

7. The crystalline Tolebrutinib:succinic acid according to claim 1, which is characterized by an X-ray powder diffraction pattern having peaks at 8.6, 9.6, 12.9, 14.2, 14.6, 16.9, 18.7, 22.4, and 24.3 degrees 2-theta±0.2 degrees 2-theta; or which is characterized by an X-ray powder diffraction pattern having peaks at 8.6, 9.6, 14.2, 14.6, 16.9, 18.7, 22.4, and 24.3 degrees 2-theta±0.2 degrees 2-theta.

8. The crystalline Tolebrutinib:succinic acid according to claim 1, designated Form S2, which is characterized by data selected from:
(a) an X-ray powder diffraction pattern substantially as depicted in FIG. 11; or
(b) an X-ray powder diffraction pattern having peaks at 3.6, 6.1 and 9.0 degrees 2-theta±0.2 degrees 2-theta.

9. The crystalline Tolebrutinib:succinic acid according to claim 1, which is characterized by an X-ray powder diffraction pattern having peaks at 3.6, 6.1, 9.0 degrees 2-theta±0.2 degrees 2-theta, and also having any one, two, three, four, five or six additional peaks selected from 11.9, 15.5, 16.0, 17.1, 18.0 and 19.5 degrees 2-theta±0.2 degrees 2-theta.

10. The crystalline Tolebrutinib:succinic acid according to claim 1, which is characterized by an X-ray powder diffraction pattern having peaks at 3.6, 6.1, 9.0, 18.0 and 19.5 degrees 2-theta±0.2 degrees 2-theta.

11. The crystalline Tolebrutinib:succinic acid according to claim 1, which is characterized by an X-ray powder diffraction pattern having peaks at 3.6, 6.1, 9.0, 18.0 and 19.5 degrees 2-theta±0.2 degrees 2-theta, and also having any one, two, three, or four additional peaks selected from 11.9, 15.5, 16.0 and 17.1 degrees 2-theta±0.2 degrees 2-theta.

12. The crystalline Tolebrutinib:succinic acid according to claim 1, which is characterized by an X-ray powder diffraction pattern having peaks at 3.6, 6.1, 9.0, 11.9, 15.5, 16.0, 17.1, 18.0 and 19.5 degrees 2-theta±0.2 degrees 2-theta.

13. The crystalline Tolebrutinib:succinic acid according to claim 1, wherein the molar ratio Tolebrutinib:succinic acid is about 2:1.

14. The crystalline Tolebrutinib:succinic acid according to claim 1, which is isolated.

15. The crystalline Tolebrutinib:succinic acid according to claim 1, which is polymorphically pure; wherein the crystalline Tolebrutinib:succinic acid contains: about 20% (w/w) or less, about 10% (w/w) or less, about 5% (w/w) or less, about 2% (w/w) or less, about 1% (w/w) or less, or about 0% of any other forms of Tolebrutinib: succinic acid.

16. The crystalline Tolebrutinib:succinic acid according to claim 1, which is enantiomerically pure, which is substantially free of the (S)-enantiomer of Tolebrutinib, wherein the crystalline Tolebrutinib:succinic acid contains: about 20% (w/w) or less, about 10% (w/w) or less, about 5% (w/w) or less, about 2% (w/w) or less, about 1% (w/w) or less, or about 0% of the (S)-enantiomer of Tolebrutinib.

17. A method comprising:
preparing using crystalline Tolebrutinib:succinic acid according to claim 1 other crystalline polymorphs of Tolebrutinib, other Tolebrutinib salts, or other Tolebrutinib cocrystals, and solid state forms thereof.

18. A method comprising:
preparing using crystalline Tolebrutinib:succinic acid according to claim 1 pharmaceutical compositions comprising Tolebrutinib or Tolebrutinib salts, or Tolebrutinib cocrystals and/or crystalline polymorphs thereof.

19. A pharmaceutical composition or formulation comprising crystalline Tolebrutinib:succinic acid according to claim 1, and at least one pharmaceutically acceptable excipient, optionally in the form of a solid dosage form, a capsule, or a tablet.

20. A process preparing a pharmaceutical composition or formulation comprising crystalline Tolebrutinib:succinic acid according to claim 1 with at least one pharmaceutically acceptable excipient.

21. The crystalline Tolebrutinib:succinic acid according to claim 1, or a pharmaceutical composition or formulation including the crystalline Tolebrutinib:succinic acid according to claim 1, for use as a medicament.

22. The crystalline Tolebrutinib:succinic acid according to claim 1, or a pharmaceutical composition or formulation including the crystalline Tolebrutinib:succinic acid according to claim 1, for use in the treatment of Multiple Sclerosis, particularly for relapsing, secondary and primary progressive Multiple Sclerosis (MS), non-relapsing secondary progressive MS, and particularly for reducing or clearing inflammation in MS brain lesions; and for the treatment of myasthenia gravis.

23. A method of treating Multiple Sclerosis, particularly for relapsing, secondary and primary progressive Multiple Sclerosis (MS), non-relapsing secondary progressive MS, and particularly for reducing or clearing inflammation in MS brain lesions; and for the treatment of myasthenia gravis, comprising administering a therapeutically effective amount of any one or a combination of a crystalline Tolebrutinib:succinic acid according to claim 1, or a pharmaceutical composition or formulation including the crystalline Tolebrutinib:succinic acid according to claim 1, to a subject in need of the treatment.
